(19) United States
(12) Patent Application Publication
(10) Pub. No.: US 2024/0238109 A1
(43) Pub. Date: Jul. 18, 2024

(54) LIMB PROSTHESIS

(71) Applicant: Koalaa Limited, London (GB)

(72) Inventors: Bryan Roberts, London (GB); Gino La, London (GB)

(21) Appl. No.: 18/429,975

(22) Filed: Feb. 1, 2024

Related U.S. Application Data
(63) Continuation of application No. PCT/IB2022/056900, filed on Jul. 26, 2022.

(30) Foreign Application Priority Data
Aug. 4, 2021 (GB) 2111233.9

(51) Int. Cl. A61F 2/80; A61F 2/50; A61F 2/58; A61F 2/78

(52) U.S. Cl. CPC A61F 2/80; A61F 2/588; A61F 2002/502; A61F 2002/5083; A61F 2002/7881

(57) ABSTRACT

The present invention relates to prostheses for limbs, particularly modular, adjustable and customisable prostheses.

### CROSS REFERENCE TO RELATED APPLICATIONS

Continuation of International Application No. PCT/IB2022/056900 filed on Jul. 26, 2022. Priority is claimed from British Application No. 2111233.9 filed on Aug. 4, 2021. Each of the foregoing applications are incorporated herein by reference in its entirety.

### STATEMENT CONCERNING FEDERALLY SPONSORED RESEARCH OR DEVELOPMENT

Not applicable.

### NAMES OF THE PARTIES TO A JOINT RESEARCH AGREEMENT

Not applicable.

### FIELD OF INVENTION

The present application relates to prosthetics for limbs.

### BACKGROUND

The partial loss or lack of part of a limb can make certain tasks more difficult. Prostheses can be used to replace at least some functions of a missing limb portion.

### SUMMARY

According to a first aspect, there is provided a prosthesis sleeve defining a longitudinal axis extending between a first end of the prosthesis sleeve and a second end of the prosthesis sleeve distal to the first end, the second end of the prosthesis sleeve being trimmable to adjust a length of the prosthesis sleeve for a user.

The second end may be hollow.

The second end may be circumferentially discontinuous. This may, for example, assist in making the sleeve usable with different limb circumferences.

The prosthesis sleeve may comprise a longitudinal opening extending from the first end towards the second end. The longitudinal opening may, for example, assist in making the sleeve usable with different limb circumferences.

The prosthesis sleeve may be configured to allow the longitudinal opening to be widened, thereby to allow a portion of a limb to be moved into the prosthesis sleeve through the widened longitudinal opening. For example, the prosthesis sleeve may be configured to allow the longitudinal opening to be widened sufficiently that the portion of a limb can be moved into the prosthesis sleeve through the widened longitudinal opening without significant shear force being applied to a distal end of the portion of a limb, optionally by moving the portion of a limb laterally.

The prosthesis sleeve may be circumferentially flexible to allow the longitudinal opening to be widened. Alternatively, or in addition, the prosthesis sleeve may comprise a hinge portion that allows the longitudinal opening to be widened.

The prosthesis sleeve may comprise a hollow cylindrical or frusto-conical body. By being hollow, the prosthesis sleeve may be kept light-weight, while the cylindrical or frusto-conical shape may, for example, assist in allowing the sleeve to conform with a limb, in use.

The prosthesis sleeve may comprise a flexible covering disposed on an outer surface.

The prosthesis sleeve may comprise a flexible liner disposed on an inner surface.

The second end may be formed from one or more materials selected such that the second end is trimmable using a hand shears or scissors. This may, for example, enable the length of the prosthesis sleeve to be conveniently customised using cheap and readily available cutting tools.

According to a second aspect, there is provided a prosthesis end cap, comprising a receiving portion configured to receive the second end of the prosthesis sleeve of any preceding claim.

The prosthesis end cap may be configured for releasable attachment to the prosthesis sleeve. This allows, for example, for the sleeve and the end cap to be separated to allow for maintenance, cleaning (such as machine washing of the sleeve), or replacement, of either component.

The receiving portion may comprise a recess. This may, for example, assist with aligning and/or supporting a prosthesis sleeve with the end cap, in use.

The recess may comprise an axial groove extending in a circumferential direction around at least a portion of the prosthesis end cap. This may, for example, retain the cut end of a prosthesis sleeve, in use, which may reduce fraying and/or reduce wear of the cut end.

The prosthesis end cap may comprise at least one hole for receiving a fastener for fastening the prosthesis end cap to the prosthesis sleeve.

The prosthesis end cap may comprise a prosthetic formation or mechanism.

The prosthesis end cap may comprise an attachment mount for releasably attaching a prosthetic formation or mechanism.

According to a third aspect, there is provided a prosthesis comprising the prosthesis end cap of the second aspect attached to the trimmed second end of the prosthesis sleeve of the first aspect.

According to a fourth aspect, there is provided a method of manufacturing the prosthesis of the third aspect, comprising:
trimming the second end of the prosthesis sleeve to adjust a length of the prosthesis sleeve for a user; and
attaching the prosthesis end cap to the trimmed second end of the prosthesis sleeve.

Trimming of the second end may comprise cutting the second end using hand shears or scissors.

Attaching the prosthesis end cap may comprise inserting the trimmed second end into an axial groove extending in a circumferential direction around at least a portion of the prosthesis end cap, and may comprise fixing the prosthesis end cap to the trimmed second end, optionally using at least one fastener.

According to a fifth aspect, there is provided a prosthesis sleeve defining a longitudinal axis extending between a first end of the prosthesis sleeve and a second end of the prosthesis sleeve distal to the first end, the prosthesis sleeve comprising at least one closure-mounting region disposed on the prosthesis sleeve, the at least one closure-mounting region being configured to releasably retain corresponding portions of at least one closure, such that the at least one closure enables cinching of the prosthesis sleeve onto a portion of a limb.

The prosthesis sleeve may comprise a longitudinal opening extending from the first end towards the second end.

The longitudinal opening may extend to the second end, such that the prosthesis sleeve is circumferentially discontinuous along its entire length. This may, for example, assist in making the sleeve usable with different limb circumferences.

The at least one closure-mounting region may extend longitudinally at or adjacent edges of the longitudinal opening. This may, for example, allow variable longitudinal and/or circumferential positioning of a closure.

The at least one closure-mounting region may cover at least 30% of an outer surface of the prosthesis sleeve. This offers a useful diversity of potential mounting positions for one or more closures while leaving at least some of the surface free of the closure-mounting region. Optionally, the at least one closure-mounting region may cover at least 60%, or even 90%, of the outer surface of the prosthesis sleeve, which may offer additional diversity of potential mounting positions for one or more closures. Diversity of potential mounting positions for one or more closures improves the ability of a user to customise prosthesis fit to their particular physical circumstances and personal preferences, which may be important given how much time the user may spend wearing a prosthesis.

The at least one closure-mounting region may take the form of a single continuous closure-mounting region.

The closure-mounting region may comprise a plurality of fasteners, the fasteners being positioned and configured to allow the at least one closure to be positioned, in use, across the longitudinal opening at any of a plurality of positions. This may, for example, allow a user to select from multiple possible positions for such a closure, which may offer improved performance.

The fasteners may comprise hook and/or loop fastener regions for retaining complementary hook and/or loop fastener regions disposed on the at least one closure. This may, for example, allow a user to position a closure relative to the closure-mounting region, which may offer improved performance.

The prosthesis sleeve may be configured to allow the longitudinal opening to be widened, thereby to allow the portion of a limb to be moved into the prosthesis sleeve through the widened longitudinal opening. For example, the prosthesis sleeve may be configured to allow the longitudinal opening to be widened sufficiently that the portion of a limb can be moved into the prosthesis sleeve laterally through the widened longitudinal opening, optionally without significant shear force being applied to a distal end of the portion of a limb.

The prosthesis sleeve may be circumferentially flexible to allow the longitudinal opening to be widened. Alternatively, or in addition, the prosthesis sleeve may comprise a hinge portion that allows the longitudinal opening to be widened.

The at least one closure-mounting region may be configured to enable releasable attachment of one or more prosthetic attachments to the prosthesis sleeve.

According to a sixth aspect, there is provided a prosthesis comprising:
the prosthesis sleeve of the fifth aspect; and
at least one closure;
the at least one closure-mounting region being configured to releasably retain corresponding portions of the at least one closure, such that the at least one closure enables cinching of the prosthesis sleeve onto a portion of a limb.

The at least one closure may comprise a strap. The strap may be configured for attachment across a longitudinal opening of the prosthesis sleeve. For example, one end of the strap may be attached via the closure-mounting region at one side of the longitudinal opening, and an opposite end of the strap may be attached via the closure-mounting region on an opposite side of the longitudinal opening.

The at least one closure may comprise an adjustable cuff.

According to a seventh aspect, there is provided a method of attaching the prosthesis of the sixth aspect to a portion of a user's limb, the method comprising the following steps, performed in any suitable order:
positioning the portion of a limb within the prosthesis sleeve;
positioning the at least one closure across the longitudinal opening;
retaining a first portion of the at least one closure at a first position of the at least one closure-mounting region; and
retaining a second portion of the at least one closure at a second position of the at least one closure-mounting region;
such that the at least one closure cinches the prosthesis sleeve onto the portion of a limb.

Positioning the portion of a limb within the prosthesis sleeve may comprise moving at least some of the portion of a limb laterally through the longitudinal opening.

The method may comprise, prior to positioning the portion of a limb within the prosthesis sleeve, widening the longitudinal opening.

The portion of a limb may comprise a partially-healed wound at its distal end, and positioning the portion of a limb within the sleeve may be performed without significant shear force being applied to the distal end of the portion of a limb.

### BRIEF DESCRIPTION OF DRAWINGS

Aspects and embodiments of the invention will now be described, by way of example only, with reference to the accompanying drawings, in which:

FIG. 1 is a perspective view of a prosthesis sleeve;

FIG. 2 is a side view of the prosthesis sleeve of FIG. 2;

FIG. 3 is a top view of the prosthesis sleeve of FIGS. 1 and 2;

FIG. 4 is a perspective view of the prosthesis sleeve of FIGS. 1-3, opened out and in the process of being trimmed to length;

FIG. 5 is a side view of the prostheses of FIGS. 1-4, showing the effect of the trimming process shown in FIG. 4;

FIG. 6 shows a cross-sectional view of a prosthesis sleeve;

FIG. 7 is a perspective view of a prosthesis end cap;

FIG. 8 is a rear perspective view of the prosthesis end cap of FIG. 7;

FIG. 9 is a sectional view through the end cap of FIGS. 7 and 8, and the prosthesis sleeve of FIGS. 1-4;

FIG. 10 is a detailed sectional view through the end cap and prosthesis of FIG. 9;

FIGS. 11 and 12 are detailed sectional views through a further end cap and prosthesis sleeve;

FIGS. 13 and 14 are detailed sectional views through yet further end caps and prosthesis sleeves;

FIGS. 15-17 show a cross-sectional sequence for installing a prosthesis onto a user's limb;

FIGS. 18 and 19 show a cross-sectional view through a further prosthesis sleeve;

FIG. 20 shows a cross-sectional view through a further prosthesis sleeve;

FIGS. 21-25 show cross-sectional views through various prosthesis sleeves;

FIG. 26 shows a method of manufacturing a prosthesis;

FIG. 27 is a perspective view of a prosthesis sleeve;

FIGS. 28-31 are top views of the prosthesis sleeve of FIG. 27 with a closure in the form of a strap;

FIGS. 32-40 are top views of further prosthesis sleeves;

FIG. 41 is a side view of a prosthesis sleeve with a closure in the form of a cuff;

FIG. 42 is a side view of the prosthesis sleeve with the cuff of FIG. 41 cinched helically around the prosthesis sleeve;

FIG. 43 is a detailed top view of a further prosthesis sleeve with a closure in the form of a strap;

FIG. 44 shows a method of attaching prosthesis to a limb portion of a user; and

FIGS. 45 and 46 are side views showing prosthesis sleeves with attachment straps.

### DETAILED DESCRIPTION

Prostheses are generally bespoke to a user, and even for a particular user may need to be adjusted as the user grows and/or their needs change. Bespoke manufacture and adjustments can be expensive and time-consuming.

The inventors have realised that long-term outcomes are improved when amputees start using prostheses earlier. There is, however, a limit on how early prostheses can be used. With a hard moulded prosthesis designed to be held against an axial end of a residual limb, wound healing must be complete and scar tissue inflammation must have receded before fitting of a prosthesis. Trying to fit a prosthesis too early can result in pain or discomfort, as well as the need for expensive adjustments to, or even replacement of, the prosthesis due to post-operative changes in the residual limb.

Referring to the drawings, there is provided a prosthesis sleeve 100 defining a longitudinal axis 102. Longitudinal axis 102 extends between a first end 104 of prosthesis sleeve 100 and a second end 106 of prosthesis sleeve 100 distal to first end 104. Prosthesis sleeve 100 is a component of a limb prosthesis, as described in more detail below.

The illustrated prosthesis sleeve 100 is for a below-elbow prosthesis. However, the skilled person will appreciate that, in other aspects, the prosthesis sleeve may be configured for use with an above-elbow prosthesis, a below-knee prosthesis, or an above-knee prosthesis.

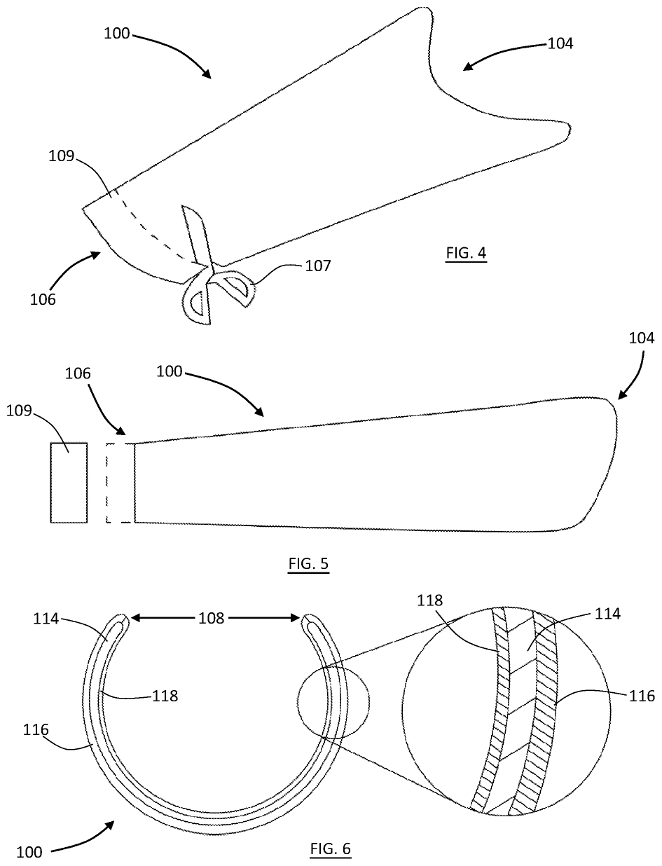

As shown in FIGS. 4 and 5, prosthesis sleeve 100 is trimmable to adjust a length of the prosthesis sleeve 100 for a user. In this context, “trimmable” means that an overall length of the prosthesis sleeve can be adjusted in a clinical setting to suit the biometrics of a specific user. For example, a relatively tall user with long limbs will typically need a longer prosthetic limb than a relatively short user with short limbs. The length of prosthesis sleeve 100 being adjustable in a clinical setting means that a single prosthesis sleeve 100, or small number of such sleeves, can be used to fit all or most potential limb sizes. This reduces the number of different-sized prosthesis sleeves 100 that must be stocked to meet the needs of all or at least most users. The length of a “trimmable” prosthesis sleeve may also be adjusted by a user without the need for a clinician being present, which may reduce fitting costs.

Prosthesis sleeve 100 comprises a hollow cylindrical or frusto-conical body. The terms “cylindrical” and “frusto-conical” are to be understood broadly rather than in a strict mathematical sense. For example, “cylindrical” includes any generally elongate volume having relatively parallel sides, although the exact cross-sectional shape and area can vary along the length of prosthesis sleeve 100, for example to suit human anatomy. Similarly, “frusto-conical” includes any generally elongate volume having a taper along some or all of its length, for example to suit human anatomy. The terms “cylindrical” and frusto-conical” are also intended to cover sleeves having walls that are not straight in longitudinal section. For example, the walls may be curved in longitudinal section, to account for curves in the limb for which the prosthesis sleeve is designed.

Second end 106 of prosthesis sleeve 100 is hollow. In this context, “hollow” means that second end 106 is not completely closed or enclosed. Depending on how this is implemented, the use of a hollow second end 106 may allow for the use of an end piece, such as the end cap described below with reference to FIGS. 7-10. Second end 106 may be hollow as a result of the construction of prosthesis sleeve 100, for example.

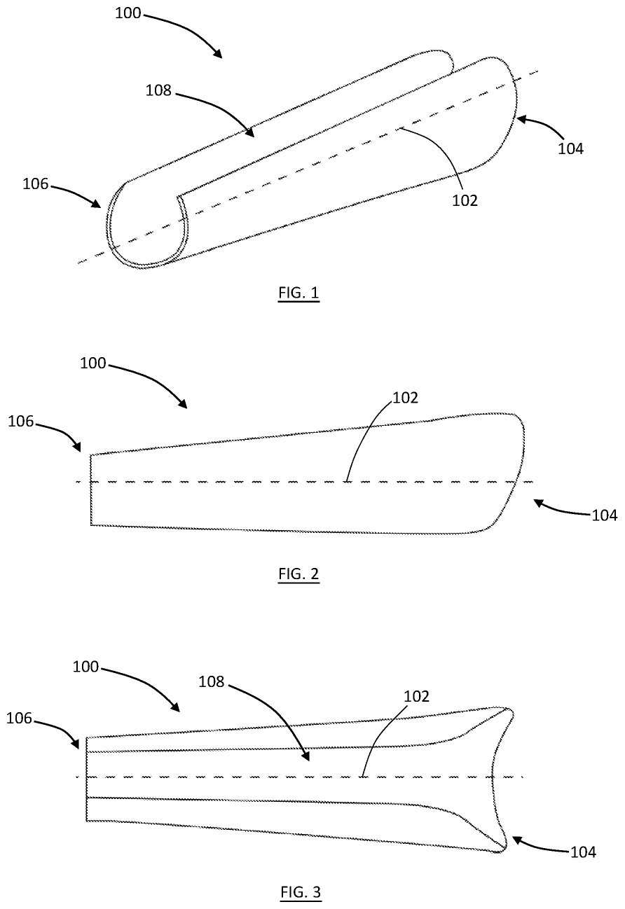

Prosthesis sleeve 100 comprises a longitudinal opening 108 extending from first end 104 towards second end 106. Longitudinal opening 108 can extend the full length of prosthesis sleeve 100, as shown in the examples of FIGS. 1-6, 15-25, 27-31, and 34-43.

Alternatively, longitudinal opening 108 can extend only part of the length of prosthesis sleeve 100. For example, as shown in FIG. 32, longitudinal opening 108 can extend from first end 104 but stop short of second end 106. This may offer, for example, a more stable second end 106, which may be useful for mounting hardware such as an end cap as described below. Alternatively, and as shown in FIG. 33, longitudinal opening 108 can extend from second end 106 but stop short of first end 104.

Prosthesis sleeve 100 can be configured to allow longitudinal opening 108 to be widened. This can allow more room within prosthesis sleeve for a portion of a limb to be inserted. Such additional room can allow for less force to be experienced by the portion of a limb, which can be important where there is an unhealed or recently healed wound that may be sensitive to shear forces, rubbing and the like.

One way of allowing longitudinal opening 108 to be widened is for prosthesis sleeve 100 to be flexible around some or all of its circumference. FIGS. 15-17 show a prosthesis sleeve 100 that is circumferentially flexible. FIG. 15 shows the prosthesis sleeve 100 in a relaxed state. FIG. 16 shows the prosthesis sleeve 100 of FIG. 15, stretched open such that longitudinal opening 108 is widened. This allows a portion of a limb 110 to be moved into prosthesis sleeve 100 through the widened longitudinal opening 108.

In the illustrated embodiment, longitudinal opening 108 can be widened sufficiently to allow the portion of a limb 110 to be moved into prosthesis sleeve 100 such that at least some of the move is made laterally. “Laterally”, in this context, means that the movement includes at least a component that is normal to the axis of sleeve 100. In some cases, “laterally” will also mean that at least some of the portion of a limb 110 passes between edges of longitudinal opening 108.

In some cases, the entire movement of the portion of a limb 110 into prosthesis sleeve 100 may be lateral, with minimal if any axial component. This may reduce or prevent shear forces being applied to the end of the user's limb, which is particularly beneficial where a wound at or adjacent a distal end of a user's limb has only partly or recently healed. This allows a user to start using a prosthesis while healing is still under way, which may be impossible or at least significantly less comfortable with stiff, axially-entered prostheses. FIG. 17 shows the prosthesis sleeve 100 of FIGS. 15 and 16, allowed to return to the relaxed state of FIG. 15, in which prosthesis sleeve 100 partly encircles the portion of a limb 110.

Another way of allowing longitudinal opening 108 to be widened is for prosthesis sleeve 100 to include at least one hinge portion. FIGS. 18 and 19 show a prosthesis sleeve 100 that includes a longitudinal hinge portion 112. Hinge portion 112 can be a mechanical hinge, or a continuous or intermittent flexible hinge formed from, for example, fabric, one or more polymeric materials, or a combination thereof. FIG. 18 shows the prosthesis sleeve 100 in a relaxed state. FIG. 19 shows the prosthesis sleeve of FIG. 18, hinged into an open position such that longitudinal opening 108 is widened.

FIG. 20 shows a prosthesis sleeve 100 that includes two longitudinal hinge portions 112. The skilled person will appreciate that three or more hinge portions 112 may be provided in other implementations.

Longitudinal opening 108 may alternatively be widened by any suitable combination of circumferential flexibility and one or more hinge portions.

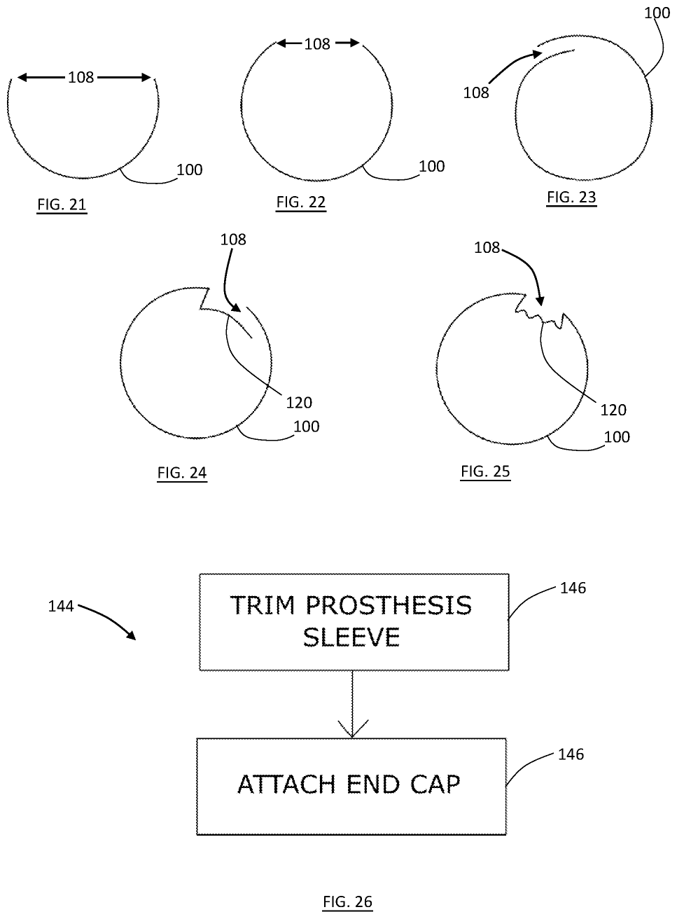

Depending on the implementation, longitudinal opening 108 can be designed to be relatively wide, as shown in FIG. 21, relatively narrow, as shown in FIG. 22 or even such that adjacent edges of prosthesis sleeve 100 adjacent opening 108 overlap as shown in FIG. 23. These widths of longitudinal opening 108 can be at rest (e.g., before installation onto a user's limb), or while prosthesis sleeve 100 is in use. Prosthesis sleeve 100 can even be designed and/or configured to be relatively flat or only slightly curved while at rest, offering ample access for a user.

If prosthesis sleeve 100 is designed such that adjacent edges of prosthesis sleeve 100 overlap, thinner flap 120 can be provided, to avoid bulkiness at the overlap. Such a flap can also be provided if the edges of the sleeve 100 do not overlap.

Prosthesis sleeve 100 may be formed from any suitable material. At least second end 106 is formed from one or more materials selected such that second end 106 is trimmable using a hand shears or (as shown in FIG. 4) scissors 107.

One example construction is shown in FIG. 6, in which prosthesis sleeve 100 comprises a core 114. Core 114 is formed from, for example, a polymer sheet. In the illustrated examples, prosthesis sleeve 100 is moulded or otherwise formed into a generally cylindrical or frusto-conical shape. In other examples (not illustrated), core 114 can take the form of a flat or only slightly curved sheet that is formed into the required cylindrical or frusto-conical shape during installation onto a user's limb. Core 114 can be formed from other materials, such as woven or nonwoven fabrics, composite materials, foils, multi-layer laminates, or any other suitable material or combination of materials.

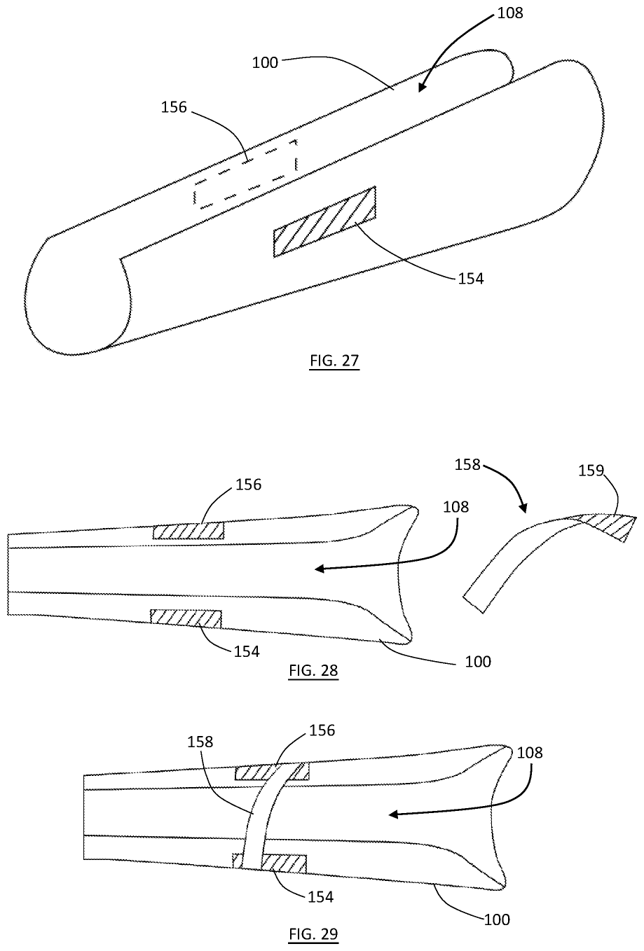

A flexible covering 116 is disposed on an outer surface of core 114 and a flexible liner 118 is disposed on an inner surface of core 114. Flexible covering 116 can be formed from any suitable material, and may optionally include an outer layer having one or more regions of hook and/or loop fastener material. Examples of such regions are described below with reference to FIGS. 27-42. Flexible liner 118 can similarly be formed from any suitable material.

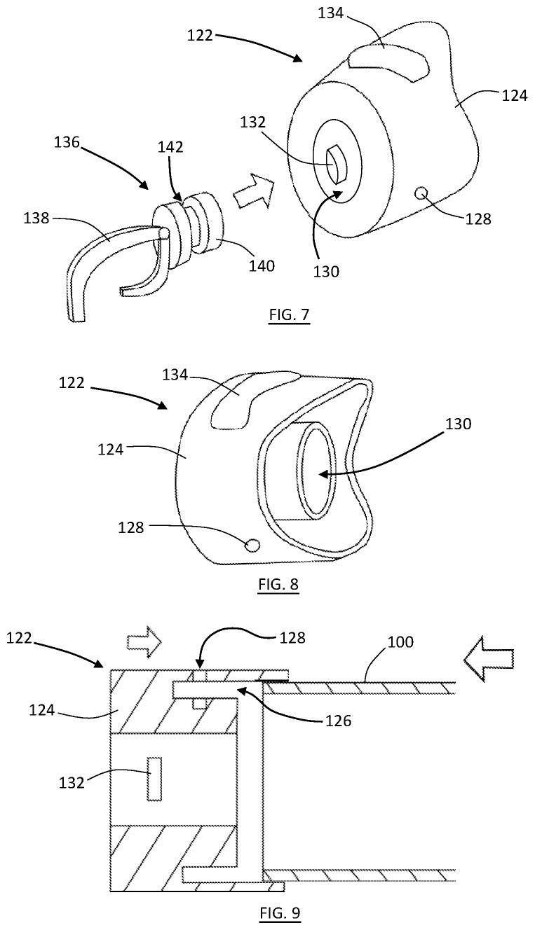

Turning to FIGS. 7 and 8, there is shown a prosthesis end cap 122. End cap 122 comprises a generally annular body 124, which comprises a receiving portion configured to receive second end 106 of the prosthesis sleeve 100. The receiving portion comprises a recess, which in the implementation of FIGS. 7 and 8, comprises an axial groove 126 extending in a circumferential direction around an axial face of prosthesis end cap 122. In the illustrated implementation, axial groove extends all of the way around prosthesis end cap 122, but in other implementations extends only partly around prosthesis end cap.

Prosthesis end cap 122 comprises several holes 128 for receiving fasteners for fastening prosthesis end cap 122 to prosthesis sleeve 100, as described in more detail below.

End cap 122 comprises a mount for a prosthetic mechanism, in the form of a socket 130. Socket 130 comprises a centrally-located hole. Retaining clips 132 extend tangentially at opposite sides of socket 130, and are biased by internal springs (not shown) towards a centre of socket 130. A release button 134 on a radially outer surface of end cap 122 is operable to cause retaining clips 132 to move radially outwardly.

Socket 130 is designed to allow for releasable mounting of a prosthetic mechanism or feature having a complimentary plug that fits within socket 130. One example of such a prosthetic mechanism is a spring-loaded pincer 136, as shown in FIG. 7. Pincer 136 includes a pair of opposed fingers 138 that are biased towards each other by a spring (not shown). Pincer 136 also includes a plug 140 that is configured to be inserted into socket 130. Plug 140 includes a peripheral groove 142 into which retaining clips 132 snap when plug 140 is inserted into socket 130. Plug 140 includes a tapered end portion allowing it to push retaining clips 132 aside as plug 140 is pushed into socket 130. Pincer 136 can be removed from end cap 122 by pushing release button 134 to move retaining clips 132 radially outward, and pulling pincer 136 out of socket 130.

The skilled person will appreciate that socket 130 can be used to mount a variety of other prosthetic formations or mechanisms.

Turning to FIG. 26, there is a shown a method 144 of manufacturing a prosthesis using the prosthesis sleeve 100 and end cap 122. It will be appreciated that both the prosthesis sleeve 100 and end cap 122 can be take many forms other than those described with reference to the accompanying drawings.

First, a second end of a prosthesis sleeve, such as prosthesis sleeve 100, is trimmed 146 to adjust a length of the prosthesis sleeve for a user, as shown in FIG. 4. The trimming can be undertaken in any suitable way. One cheap and convenient way of trimming is to use hand shears or scissors, although any other suitable method of cutting may be used depending upon the materials to be trimmed. FIG. 4 shows scissors 107 being used to trim second end 106 of prosthesis sleeve 100, which involves cutting off a piece 109.

Next, a prosthesis end cap, such as end cap 122, is attached 148 to the trimmed second end of prosthesis sleeve 100, to form a prosthesis. The attachment can be made in any suitable way. Attachment can be made in a permanent manner, such as with an adhesive, rivets or other permanent attachment method. Attachment can also be made in a non-permanent manner, such as with screws, clamps or other non-permanent attachment method.

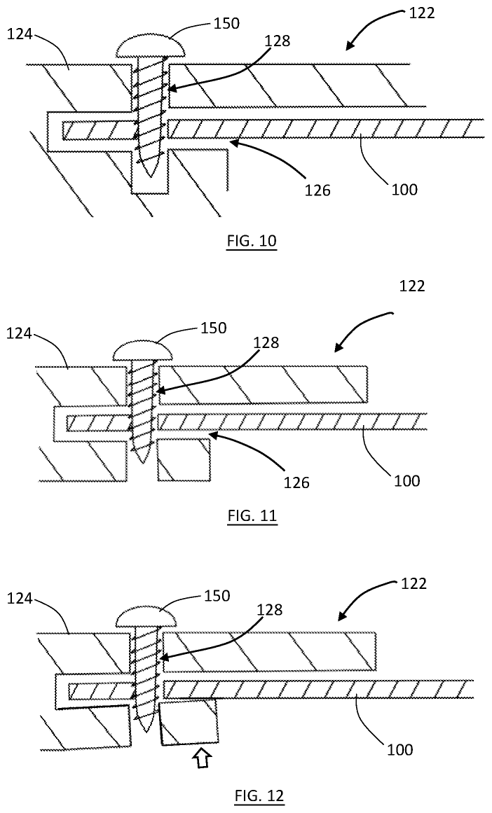

One way in which the attachment can be made is shown in FIGS. 9 and 10. Second end 106 of prosthetic sleeve 100 is axially inserted into groove 126. Fasteners in the form of self-tapping screws 150 are screwed through holes 128 in end cap 122. As best shown in FIG. 10, the tip of each screw 150 bores through prosthesis sleeve 100 and then into a radially inner portion of end cap 122. While one or two screws 150 may be sufficient to hold end cap 122 in place on prosthesis sleeve, addition screws help hold the end cap 122 in a more stable position, which may be particularly useful where a prosthetic mechanism or attachment is to be used.

Inserting the trimmed end of prosthesis sleeve 100 into groove 126 of end cap 122 helps neaten the trimmed end and reduces fraying and wear. This may be particularly useful when there is an inner and/or outer fabric or other thin material layer that would otherwise be subject to wear.

An alternative to the attachment arrangement of FIGS. 9 and 10 is shown in FIGS. 11 and 12. In the arrangement of FIGS. 11 and 12, inner portion 152 adjacent groove 126 is flexible. As screw 150 is tightened, inner portion 152 is drawn radially outwards, causing groove 126 to narrow until the trimmed end of prosthesis sleeve 100 is clamped within groove 126 by inner portion 152.

Cleats, ribs, or other gripping formations (not shown) can be added to improve retention of the prosthesis sleeve by end cap 122.

It will be appreciated that, in other implementations, the annular portions radially inward and/or outward of groove 126 may be discontinuous. For example, the inner annular portion may take the form of axially extending fingers rather than a continuous annular ring. The outer annular portion may be similar, although for reasons of neatness and protection from wear and fraying, it may be preferable to use a continuous outer annular portion.

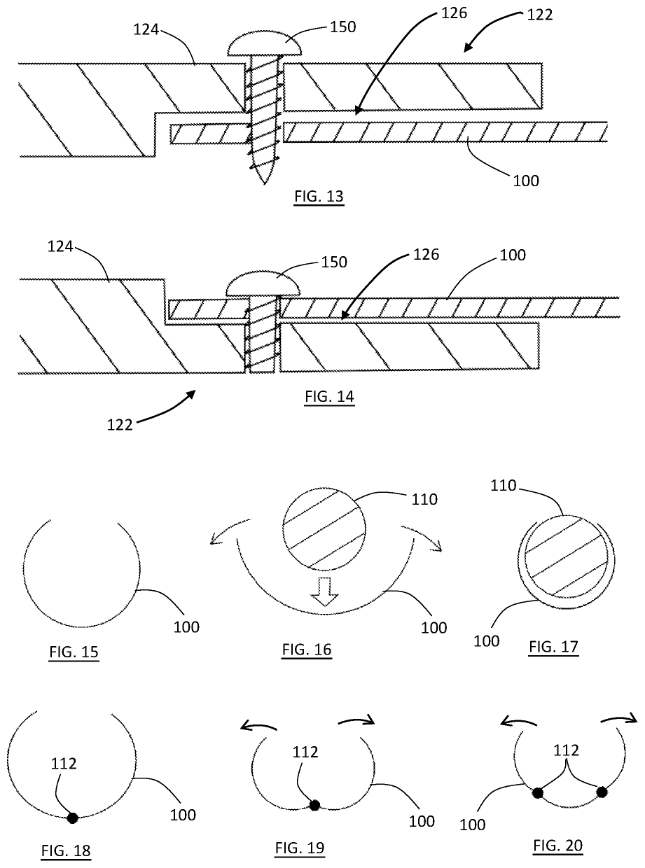

Alternative implementations are shown in FIGS. 13 and 14, in which an annular recess is formed on the inner surface (FIG. 13) or outer surface (FIG. 14), rather than in an axial surface of end cap 122.

Turning to FIGS. 27 to 44, there are shown various implementations of prosthesis sleeves, in which previously described elements are indicated with like reference signs. Optionally, each of the prosthesis sleeves in FIGS. 27 to 43 may be trimmable and usable with an end cap as described above in relation to FIGS. 1 to 24.

FIGS. 27-31 show a prosthesis sleeve 100 having a first closure-mounting region 154 and a second closure-mounting region 156. First closure-mounting region 154 and second closure-mountain region 156 comprise a receptive surface, in the form of hook/loop surface for use with a complementary loop/hook surface. The receptive surface may take the form of a fabric that is stitched, bonded, welded, and/or otherwise attached to an outer surface of prosthesis sleeve 100. Alternatively, the hooks and/or loops of the receptive surface may be woven or otherwise formed directly into or onto a fabric or other material forming the outer surface of prosthesis sleeve 100.

In FIGS. 27-31, first and second closure-mounting regions 154 and 156 are approximately in line with each other in the longitudinal direction, and extend longitudinally along prosthesis sleeve 100.

Portions of the first and second closure-mounting regions 154 and 156 are configured to releasably retain corresponding portions of at least one closure. In the example of FIGS. 27 to 31, the closure takes the form of a strap 158, one side 159 of which is covered with a loop/hook surface that is complementary to the hook/loop surface of the first and second closure-mounting regions 154 and 156. Alternatively, only part or parts of strap 158 have the required loop/hook regions.

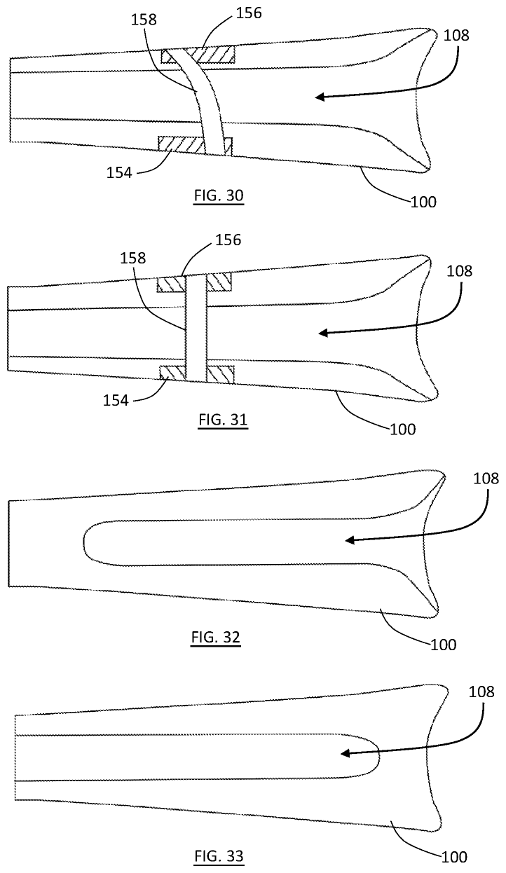

In use, once a user's limb is at least partly encircled by prosthesis sleeve 100 (see, for example, FIG. 17), strap 158 can be positioned across the longitudinal opening and attached to the first and second closure-mounting regions 154 and 156 to enable cinching of the prosthesis sleeve onto a portion of the limb. As shown in FIGS. 29 and 30, the longitudinal extents of the first and second closure-mounting regions 154 and 156 enable both ends of strap 158 to be selectively positioned at the most suitable position relative to the first and second closure-mounting regions 154 and 156. This allows a user to position strap 158 taking into account their physical needs and the needs of any particular activity for which the prosthesis is to be employed. For example, a user may prefer to place strap 158 in one position for activities such as drawing, and in a different position for more strenuous activities such as sport.

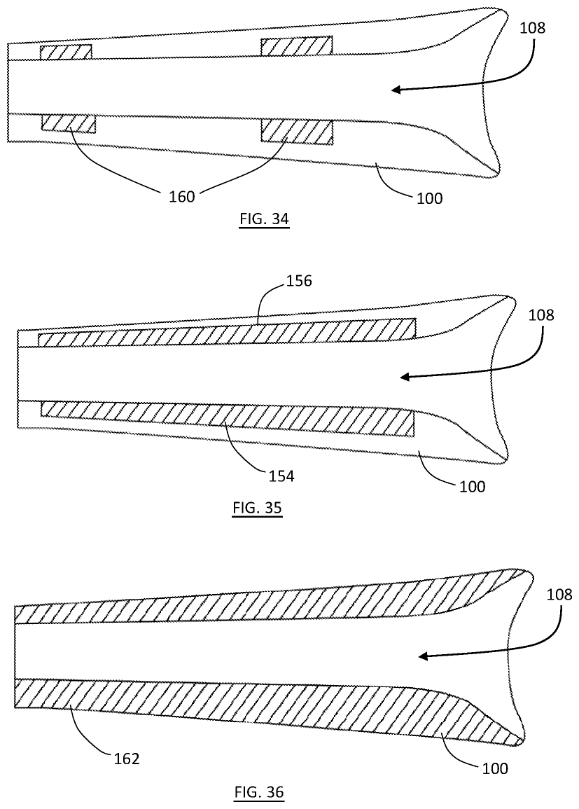

FIG. 34 shows a prosthesis sleeve 100 having four closure-mounting regions 160. Each of closure-mounting regions 160 is similar to the first and second closure-mounting regions 154 and 156 of FIGS. 27 to 31. In the case of FIG. 34, however, closure-mounting regions 160 are positioned in pairs that are longitudinally spaced apart. This enables the use of closures, such as strap 158, at a wider range of positions along prosthesis sleeve 100. The skilled person will appreciate that the closure-mounting regions 160 may be positioned further away from longitudinal opening 108 than shown in FIG. 34, and in particular need not be longitudinally aligned with each other.

FIG. 35 shows a prosthesis sleeve 100 having first and second closure-mounting regions 154 and 156. In contrast to the first and second closure-mounting regions 154 and 156 of FIGS. 27 to 30, the first and second closure-mounting regions 154 and 156 of FIG. 35 extend substantially the entire length of prosthesis sleeve 100. This enables an even wider range of positions for closures, such as strap 158, along prosthesis sleeve 100. The greater surface area also allows for the potential use of wider and/or additional closures, which may allow a user to select from a greater range of closure orientation types and positions.

The at least one closure-mounting region can cover at least 30% of an outer surface of the prosthesis sleeve. The greater the surface area covered by the at least one closure-mounting region, the greater the range of options for positioning closures such as straps 158. Optionally, the at least one closure-mounting region covers at least 60%, or even 90%, of an outer surface of the prosthesis sleeve, which further increase the range of options for positioning closures such as straps 158.

FIG. 36 shows a prosthesis sleeve 100 having a single continuous closure-mounting region 162 covering substantially all of the outer surface of prosthesis sleeve 100. This maximises the range of possible closure types and closure positions, since a user can position closures anywhere on prosthesis sleeve, both longitudinally and circumferentially. This approach may also simplify construction of prosthesis sleeve 100, because there is no need to cut and attach separate closure-mounting regions at particular positions on prosthesis sleeve 100. While more receptive material is required to cover substantially all of prosthesis sleeve 100, the reduction in labour may offset the additional material cost, or even result in a cheaper overall product, as well as enhancing functionality.

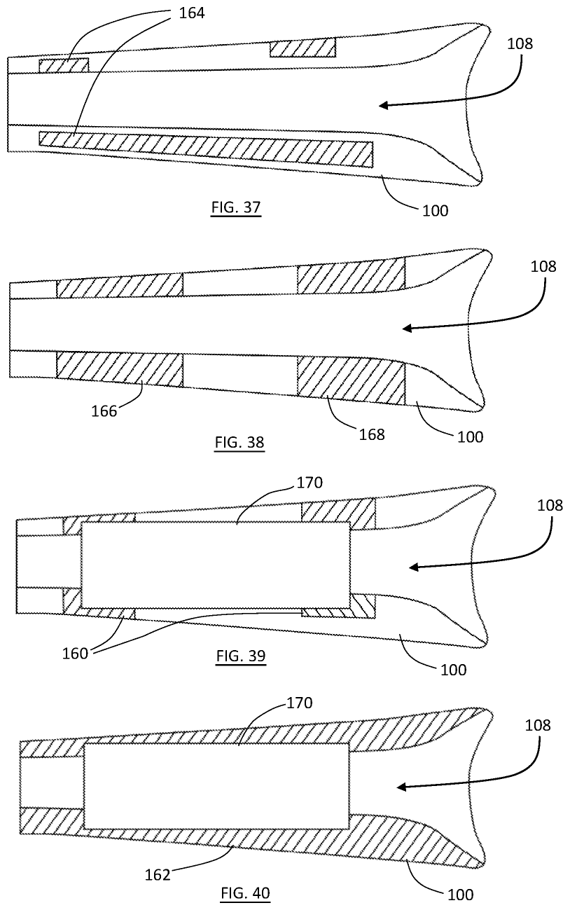

FIG. 37 shows a prosthesis sleeve 100 having several closure mounting regions 164, showing how different sizes and positions of closure mounting regions can be combined on a single prosthesis sleeve. In this case, one of closure mounting regions 164 extends along substantially the full length of prosthesis sleeve 100, whereas the remaining closure mounting regions 164 are positioned on the other side of longitudinal opening 108.

FIG. 38 shows a prosthesis sleeve 100 having two spaced apart closure-mounting regions 166 and 168, both of which encircle prosthesis sleeve 100.

FIG. 39 shows a prosthesis sleeve 100 similar to that of FIG. 34, but using a different closure in the form of a sheet 170. Like strap 158, sheet 156 has a loop/hook surface that is complementary to the hook/loop surface of closure-mounting regions 160. Sheet 170 covers a significant portion of longitudinal opening 108, which covers and helps protect the user's limb. The additional surface area of the attachment to closure-mounting regions 160 also provides additional support and stability to the prosthesis as a whole.

FIG. 40 shows a prosthesis sleeve 100 corresponding to that in FIG. 36, but using a closure in the form of sheet 170. In this case, sheet 170 is effectively attached to closure-mounting region 162 along the full length of sheet 170. This provides more attached surface area than a narrow strap, such as strap 158. Sheet 170 may therefore be used where additional support is required, such as when playing sport, and/or where additional protection for the limb is desired by the user.

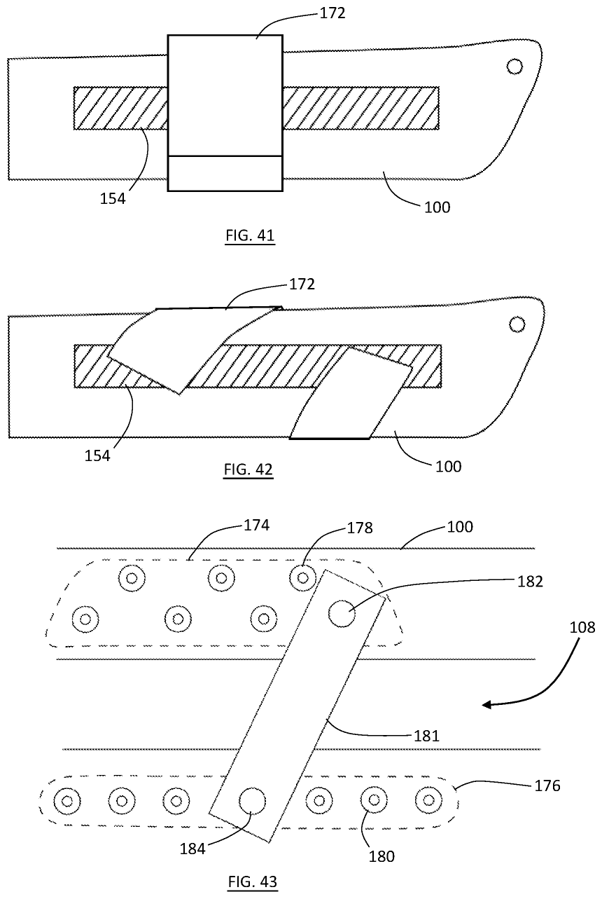

It will be appreciated that the closure may take forms other than strap 158 and sheet 170. For example, a longer strap can be used, that wraps all the way around prosthesis sleeve 100. For example, as shown in FIG. 41, a longer strap can take the form of a cuff 172 that, in use, encircles prosthesis sleeve 100. Cuff 172 can be held in longitudinal position relative to prosthesis sleeve by having a fastener that fastens to closure-mounting region 154. For example, if closure mounting region 154 comprises a receptive surface such as loop fabric, cuff 172 can include complementary hook fabric on its inner surface.

Cuff 172 can also include further fasteners and/or fastening regions enabling it to releasably cinch around prosthesis sleeve 100. Those fasteners and/or fastening regions can include hook/loop fasteners, snap-fasteners, clips, mechanical or electronic lacing system, or any other suitable type of fastener.

Optionally, cuff 172 can be used in one or more modes other than strictly as a cuff. For example, cuff 172 can be configured such that it winds helically around prosthesis sleeve 100, as shown in FIG. 42.

While several examples describe the use of closure-mounting regions that rely on receptive surfaces such as hook and/or loop surfaces, the skilled person will appreciate that many other forms of fastener may be employed. In general, the closure-mounting region typically comprises a plurality of fasteners, the fasteners being positioned and configured to allow the at least one closure to be positioned, in use, across the longitudinal opening at any of a plurality of positions. Where hook and loop fastening is employed, each potential hook and eye pair can be thought of as a fastener.

One example that does not use hook and loop surfaces is shown in FIG. 43, in which prosthesis sleeve 100 includes a first closure-mounting region 174 and a second closure-mounting region 176. First closure-mounting region 174 includes an array of spaced-apart male snap-fasteners 178, and second closure-mounting region 176 includes an array of spaced-apart male snap-fasteners 180. A closure in the form of strap 181 includes a first female snap-fastener 182 and a second female snap-fastener 184. By connecting first female snap fastener 182 to a selected one of male snap-fasteners 178, and second female snap-fastener 184 to a selected one of male snap-fasteners 180, a user can adjust the longitudinal position of strap 181 relative to prosthesis sleeve 100. Choosing different combinations of first and second male snap-fasteners 178 and 180 also allows a user to cinch prosthesis sleeve 100 to a limb with differing degrees of force, depending upon comfort and the application to which prosthesis sleeve is to be put. This also allows adjustment of a longitudinal position of strap 181.

The skilled person will appreciate that the first and second male snap-fasteners 178 and 180 can be provided in greater or lesser number than shown, and can be arranged in any suitable fashion. Similarly, strap 158 can include additional female snap-fasteners, thereby offering yet more options for cinching prosthesis 100 to a limb with differing degrees of force and in different positions (including longitudinal positions) as required. Also, any suitable combination of male and female snap-fasteners may be employed to suit user requirements, and can be combined with hook and loop fasteners and any other type of fastener.

As with the earlier-described trimmable implementations, the prosthesis sleeve can be configured to allow the longitudinal opening to be widened, thereby to allow the portion of a limb to be moved into the prosthesis sleeve laterally through the widened longitudinal opening. Optionally, the longitudinal opening can be widened sufficiently that the portion of a limb can be moved into the prosthesis sleeve laterally through the widened longitudinal opening without significant shear force being applied to a distal end of the portion of a limb.

Similarly, the prosthesis sleeve can be circumferentially flexible to allow the longitudinal opening to be widened, and can optionally comprise one or more hinge portions that allow the longitudinal opening to be widened.

The at least one closure-mounting region can be configured to enable releasable attachment of one or more prosthetic attachments to the prosthesis sleeve. For example, pincers, a clamp, a prosthetic formation, or any other prosthetic attachment can be completely or partly supported by releasable attachment to the at least one closure-mounting region.

Optionally, prosthesis sleeve 100 can include one or more mounting points for attachment straps and the like. For example, as shown in FIG. 45, an attachment strap 196 can be attached to corresponding mounting points in the form of snap fasteners 198. Alternatively, hook and loop fastening can be used. For example, as shown in FIG. 46, the attachment strap 196 can be attached to prosthesis sleeve 100 by way of a hook and/or loop surface 200. Optionally, the mounting points can form part of the at least one closure-mounting region.

Any of the described straps and closures can be formed from suitable material or materials. Examples include woven nylon, polypropylene, and cotton. Synthetic materials may be preferable due to their durability and stain resistance. Optionally, any of the described straps and closures can be at least partly constructed from a stretch or elasticated material, such as an elastane fabric. The use of a stretchable fabric allows for a more resilient cinching, which may offer a more comfortable and/or secure arrangement.

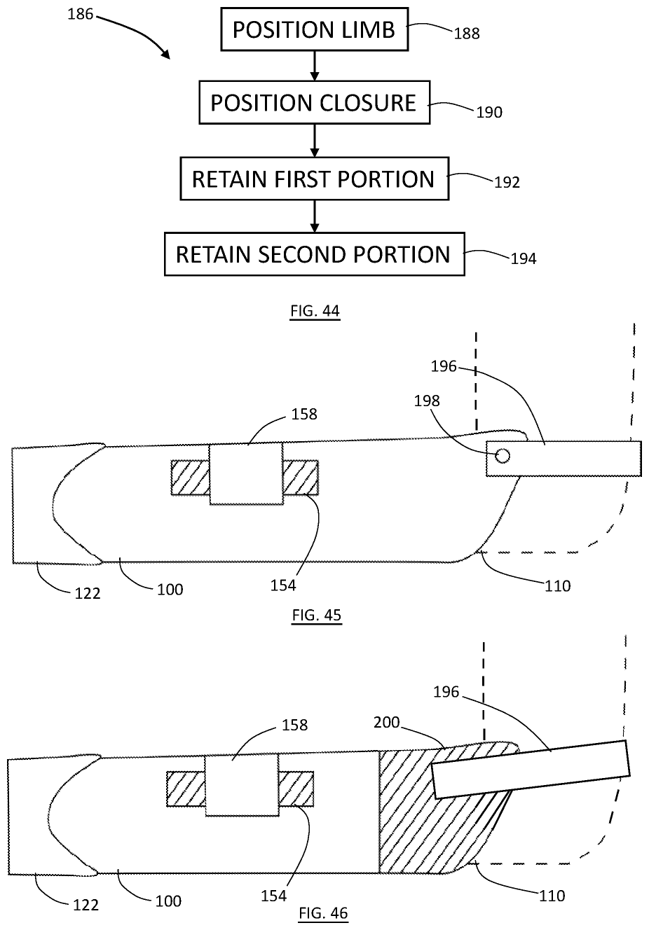

Turning to FIG. 44, there is shown a method 186 of attaching a prosthesis to a limb portion of a user. Although shown as a flowchart, the skilled person will appreciate that the steps may be performed in any suitable order.

First, a portion of a limb is positioned 188 within the prosthesis sleeve. The prosthesis sleeve can, for example take the form of any of the prosthesis sleeve is described or claimed herein, and includes any such prosthesis sleeve forming part of a prosthesis.

At least one closure is positioned 190 across the longitudinal opening.

A first portion of the at least one closure is retained 192 at a first position of the at least one closure-mounting region, and a second portion of the at least one closure is retained 194 at a second position of the at least one closure-mounting region. “Position”, in this context, means any longitudinal or circumferential position. Optionally, each position is at least partly defined by a longitudinal component, and is selected from a plurality of possible longitudinal positions.

The result of the method is that the at least one closure cinches the prosthesis sleeve onto the portion of a limb.

Optionally, positioning the portion of a limb within the prosthesis sleeve comprises moving at least some of the portion of a limb laterally through the longitudinal opening. Especially when the longitudinal opening is widened sufficiently before this takes place, little or no shear force is applied to the end of the user's limb, which is particularly beneficial where a wound at or adjacent a distal end of a user's limb has only partly or recently healed.

Prior to positioning the portion of a limb within the prosthesis sleeve, the longitudinal opening may be widened. In some implementations, the longitudinal opening in the prosthesis sleeve's resting state is wide enough that no separate widening step is required. Alternatively, the longitudinal opening may be widened to the required degree before inserting the limb.

The described implementations have been intended for use by people with below-elbow limb loss or reduced limb function, and there are particular advantages that apply to that particular application. However, the skilled person will appreciate that other implementations can be applied to other situations in which there is a loss of, or reduction in function of, a limb, including above or below-knee, and above-elbow.

Although various specific implementations have been described, the skilled person will appreciate that the invention may be embodied in many other forms.

Approximately as employed herein means+10%.

In the context of this specification “comprising” is to be interpreted as “including”.

Aspects of the invention comprising certain elements are also intended to extend to alternative embodiments “consisting” or “consisting essentially” of the relevant elements.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

What is claimed is:

1. A prosthesis sleeve defining a longitudinal axis extending between a first end of the prosthesis sleeve and a second end of the prosthesis sleeve distal to the first end, the second end of the prosthesis sleeve being trimmable to adjust a length of the prosthesis sleeve for a user.

2. The prosthesis sleeve of claim 1, wherein the second end is hollow.

3. The prosthesis sleeve of claim 1, wherein the second end is circumferentially discontinuous.

4. The prosthesis sleeve of claim 1, comprising a longitudinal opening extending from the first end towards the second end.

5. The prosthesis sleeve of claim 4, configured to allow the longitudinal opening to be widened, thereby to allow a portion of a limb to be moved into the prosthesis sleeve laterally through the widened longitudinal opening.

6. The prosthesis sleeve of claim 5, configured to allow the longitudinal opening to be widened sufficiently that the portion of a limb can be moved into the prosthesis sleeve laterally through the widened longitudinal opening without significant shear force being applied to a distal end of the portion of a limb.

7. The prosthesis sleeve of claim 5, wherein the prosthesis sleeve is circumferentially flexible to allow the longitudinal opening to be widened.

8. The prosthesis sleeve of claim 5, wherein the prosthesis sleeve comprises a hinge portion that allows the longitudinal opening to be widened.

9. The prosthesis sleeve of claim 1, comprising a hollow cylindrical or frusto-conical body.

10. The prosthesis sleeve of claim 1, comprising a flexible covering disposed on an outer surface.

11. The prosthesis sleeve of claim 1, comprising a flexible liner disposed on an inner surface.

12. The prosthesis sleeve of claim 1, wherein the second end is formed from one or more materials selected such that the second end is trimmable using a hand shears or scissors.

13. A prosthesis end cap, comprising a receiving portion configured to receive the second end of the prosthesis sleeve of claim 1.

14. The prosthesis end cap of claim 13, configured for releasable attachment to the prosthesis sleeve.

15. The prosthesis end cap of claim 13, wherein the receiving portion comprises a recess.

16. The prosthesis end cap of claim 15, wherein the recess comprises an axial groove extending in a circumferential direction around at least a portion of the prosthesis end cap.

17. The prosthesis end cap of claim 13, comprising at least one hole for receiving a fastener for fastening the prosthesis end cap to the prosthesis sleeve.

18. The prosthesis end cap of claim 13, comprising a prosthetic formation or mechanism.

19. The prosthesis end cap of claim 13, comprising an attachment mount for releasably attaching a prosthetic formation or mechanism.

20. A prosthesis comprising the prosthesis end cap of claim 13, attached to the trimmed second end of the prosthesis sleeve of claim 1.

21. A method of manufacturing the prosthesis of claim 20, comprising:
trimming the second end of the prosthesis sleeve to adjust a length of the prosthesis sleeve for a user; and
attaching the prosthesis end cap to the trimmed second end of the prosthesis sleeve.

22. The method of claim 21, wherein trimming the second end comprises cutting the second end using hand shears or scissors.

23. The method of claim 21, wherein attaching the prosthesis end cap comprises:
inserting the trimmed second end into an axial groove extending in a circumferential direction around at least a portion of the prosthesis end cap; and
fixing the prosthesis end cap to the trimmed second end using at least one fastener.

24. A prosthesis sleeve defining a longitudinal axis extending between a first end of the prosthesis sleeve and a second end of the prosthesis sleeve distal to the first end, the prosthesis sleeve comprising at least one closure-mounting region disposed on the prosthesis sleeve, the at least one closure-mounting region being configured to releasably retain corresponding portions of at least one closure, such that the at least one closure extends across the longitudinal opening to enable cinching of the prosthesis sleeve onto a portion of a limb.

25. The prosthesis sleeve of claim 24, comprising a longitudinal opening extending from the first end towards the second end.

26. The prosthesis sleeve of claim 25, wherein the longitudinal opening extends to the second end, such that the prosthesis sleeve is circumferentially discontinuous along its entire length.

27. The prosthesis sleeve of claim 25, wherein the at least one closure-mounting region extends longitudinally at or adjacent edges of the longitudinal opening.

28. The prosthesis sleeve of claim 25, wherein the closure-mounting region comprises a plurality of fasteners, the fasteners being positioned and configured to allow the at least one closure to be positioned, in use, across the longitudinal opening at any of a plurality of positions.

29. The prosthesis sleeve of claim 28, wherein the fasteners comprise hook and/or loop fastener regions for retaining complementary hook and/or loop fastener regions on the at least one closure.

30. The prosthesis sleeve of claim 24, configured to allow the longitudinal opening to be widened, thereby to allow the portion of a limb to be moved into the prosthesis sleeve laterally through the widened longitudinal opening.

31. The prosthesis sleeve of claim 30, configured to allow the longitudinal opening to be widened sufficiently that the portion of a limb can be moved into the prosthesis sleeve laterally through the widened longitudinal opening without significant shear force being applied to a distal end of the portion of a limb.

32. The prosthesis sleeve of claim 30, wherein the prosthesis sleeve is circumferentially flexible to allow the longitudinal opening to be widened.

33. The prosthesis sleeve of claim 30, wherein the prosthesis sleeve comprises a hinge portion that allows the longitudinal opening to be widened.

34. The prosthesis sleeve of claim 24, wherein the at least one closure-mounting region covers at least 30%, and optionally at least 60%, of an outer surface of the prosthesis sleeve.

35. The prosthesis sleeve of claim 24, wherein the at least one closure-mounting region takes the form of a single continuous closure-mounting region.

36. The prosthesis sleeve of claim 24, wherein the at least one closure-mounting region is configured to enable releasable attachment of one or more prosthetic attachments to the prosthesis sleeve.

37. A prosthesis comprising:
the prosthesis sleeve of claim 24; and
at least one closure;
the at least one closure-mounting region being configured to releasably retain corresponding portions of the at least one closure, such that the at least one closure enables cinching of the prosthesis sleeve onto a portion of a limb.

38. The prosthesis of claim 37, wherein the at least one closure comprises a strap.

39. The prosthesis of claim 37, wherein the at least one closure comprises an adjustable cuff.

40. A method of attaching the prosthesis of claim 26 to a limb portion of a user, the method comprising the following steps, performed in any suitable order:
positioning the portion of a limb within the prosthesis sleeve;
positioning the at least one closure across the longitudinal opening;
retaining a first portion of the at least one closure at a first position of the at least one closure-mounting region; and
retaining a second portion of the at least one closure at a second position of the at least one closure-mounting region;
such that the at least one closure cinches the prosthesis sleeve onto the portion of a limb.

41. The method of claim 40, wherein positioning the portion of a limb within the prosthesis sleeve comprises laterally moving at least some of the portion of a limb laterally through the longitudinal opening.

42. The method of claim 40, comprising, prior to positioning the portion of a limb within the prosthesis sleeve, widening a longitudinal opening of the prosthesis sleeve.

43. The method of claim 40, wherein:
the portion of a limb comprises a partially-healed wound at its distal end; and
positioning the portion of a limb within the sleeve is performed without significant shear force being applied to the distal end of the portion of a limb.
